(19) United States
(12) Patent Application Publication
(10) Pub. No.: US 2005/0025736 A1
(43) Pub. Date: Feb. 3, 2005

(54) Hair and skin altering and protecting compositions

(76) Inventors: Janusz Jachowicz, Bethel, CT (US); Roger L. Mc Mullen JR., Bloomfield, NJ (US); Linda C. Foltis, Nutley, NJ (US); Jean Karolak, Wayne, NJ (US); Steve Orofino, Hamburg, NJ (US); Blanca Gomez, Newark, NJ (US); Ellen S. Botschka, Riverdale, NJ (US)

Correspondence Address: INTERNATIONAL SPECIALTY PRODUCTS;Attn: William J. Davis, Esq., Legal Department, Building No. 10, 1361 Alps Road, Wayne, NJ, 07470, US

(21) Appl. No.: 10/630,560

(22) Filed: Jul. 30, 2003

(51) Int. Cl. A61K 7/06; A61K 7/11; A61K 7/13

(52) U.S. Cl. 424/70.15; 424/70.17; 8/406

(57) ABSTRACT

What is described herein are compositions for treating hair and skin containing a cationic polymer derived from an N-vinyl lactam, preferably vinyl pyrrolidone (VP), a dimethylaminoalkylmethacrylamide, preferably dimethylaminopropyl methacrylamide (DMAPMA) and a quaternized C₉ to C₂₄ alkyl dimethylaminoalkyl(meth)acrylamide, preferably quaternized dimethylaminopropyl(meth)acrylamide (QDMAPMA) in a cosmetically acceptable concentration range. Hair, skin and other personal care compositions which include these and similar polymers described herein exhibit low tackiness, superior fixative properties and high humidity resistance as well as excellent penetrating and conditioning properties for oxidizing or reducing compositions as required in hair coloring, bleaching and permanent waving while further promoting hair texture and shine.

### BACKGROUND OF THE INVENTION

1. Field of the Invention

This invention relates to compositions for use in hair and skin care featuring a specific cationic polymer, and, more particularly, to conditioning and styling compositions advantageously having improved conditioning, low tackiness and high humidity resistance.

2. Description of the Prior Art

Compositions containing copolymers derived from vinylpyrrolidone (VP) and dimethylaminopropyl methacrylamide (DMAPMA) have been used extensively as active components of hair and skin compositions. While these copolymers are generally suitable polymers for such products as conditioners and shampoos, it is desired to provide superior polymers having improved performance characteristics in these and other personal care products.

Commonly-owned U.S. Pat. No. 6,207,778 discloses conditioning/styling terpolymers of VP, DMAPMA and C₉-C₂₄ alkyl dimethylaminopropyl methacrylic acid quaternized monomers (QDMAPMA) and hair and skin care compositions which include these terpolymers and exhibit low tackiness and high humidity resistance.

It is generally recognized that hair and skin formulations contain a mixture of components, including one or more of those selected from the group consisting of conditioning agents, protecting agents, such as, for example, hydrosoluble, liposoluble and water-insoluble UV filters, antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, hydroxy acids, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These components usually comprise 0.001 to 20 wt. % of the total formulation. The disclosures of international publications WO 02/058661, pages 7-24; WO 02/058660, pages 7-8, WO 02/058649 at pages 6-13; WO 02/058648 at pages 6-26 and WO 02/058646 provide examples of such known and conventionally employed hair and skin formulation additives, typical use concentrations and suppliers thereof or identification of references where they are described. The disclosures of WO 02/058661, WO 02/058660, WO 02/058649, WO 02/058648 and WO 02/058646 are hereby incorporated by reference in their entirety.

### SUMMARY OF THE INVENTION

In accordance with this invention there is provided various personal care compositions and formulations which include conventional concentrations of known hair and skin altering components and a cosmetically acceptable amount of a cationic polymer derived from monomers of a vinyl lactam, an aminoalkyl acrylamide and/or an aminoalkyl acrylate, and a quaternized aminoalkyl acrylamide and/or a quaternized aminoalkyl acrylate and mixtures thereof.

For the purposes of this disclosure, the term “acrylic” is intended to include unsubstituted acrylic and methacrylic radicals. Similarly “acrylamide” and “acrylate” are intended to include methacrylamide and methacrylate.

More specifically, in accordance with this invention there is provided a personal care composition for treatment of hair or skin comprising:
(i) a cosmetically acceptable medium comprising at least one cosmetically acceptable additive selected from conditioning agents, skin enhancing agents, protecting agents, fixing agents, oxidizing agents, reducing agents and dyes, and
(ii) a cationic polymer derived from:
   (a) at least one vinyllactam or alkylvinyllactam monomer;
   (b) at least one monomer having the structure 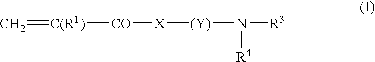
   and
   (c) at least one quaternized monomer having the structure 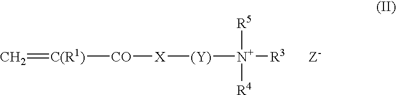
   wherein X is O or NR⁶;
   R¹ and R⁶ are each independently H or C₁-C₅ alkyl;
   R³, R⁴ and R⁵ are each independently H or C₁-C₃₀ alkyl;
   Y is an alkylene radical having from 2 to 16 carbon atoms and
   Z is an anion of an organic or mineral acid.

### DETAILED DESCRIPTION OF THE INVENTION

The cationic polymers employed in the compositions of the invention preferably comprise the following X, I, and II monomers, X having the formula: 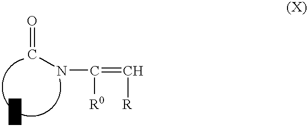
wherein
X is an N-aliphatically unsaturated cyclic amide, e.g. vinyl pyrrolidone or vinyl caprolactam, P is C₃ to C₆ alkylene optionally substituted with methyl or ethyl, R and R^{o} are each independently H or C₁-C₅ alkyl and where the weight % concentration of X in the polymer is between about 40-95; preferably 60-90%;
I is a derivative of an acrylic acid and where the weight % concentration of I in the polymer is between about 0.1 to 55; preferably 5-30%; and
II is a quaternized derivative of an acrylic acid in which Z is an organic or mineral acid anion, e.g., a methosulfate, halide, tosylate or phosphate anion and where the weight % concentration of 11 in the terpolymer is between about 0.25 to 50, preferably 0.50 to 40 and most preferably 1-30%.

The cationic polymers employed in the compositions of the invention are hydrophobically-modified cationic polymers having at least one long chain alkyl radical in either or both of monomers 1 and 11. A typical and preferred cationic polymer is a terpolymer composed of the following recurring units:

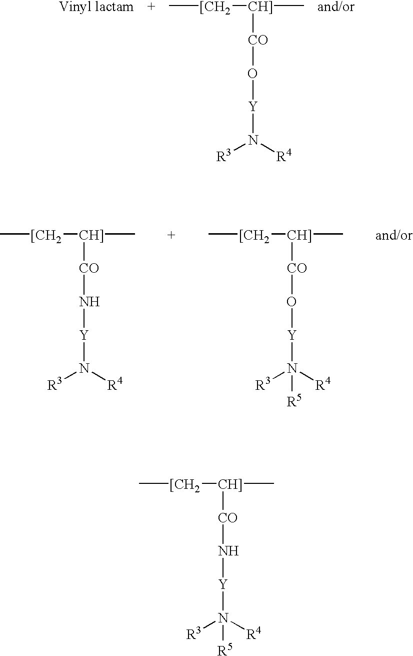

Most preferred is the terpolymer derived from N-vinyl pyrrolidone, dimethylaminopropyl methacrylamide and a quaternized salt of dimethylaminopropyl methacrylamide, i.e. (VP/DMAPMA/QDMAPMA).

Suitably, monomer 11 can be prepared by quaternizing, e.g. tosylating, the corresponding acrylate or acrylamide; by way of illustration: 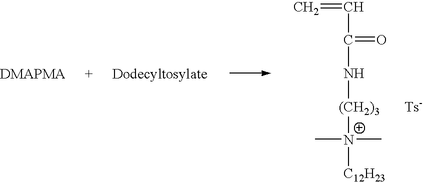
where Ts is the tosylate anion of dodecyl tosylate. A more complete disclosure of applicants' preferred cationic polymer and its preparation is described in commonly owned U.S. Pat. No. 6,207,778 and in U.S. Pat. No. 5,626,836, the disclosures of which are hereby incorporated herein by reference in their entirety.

In a preferred embodiment of the present invention, X is N-vinyl pyrrolidone, I is dimethylaminopropyl methacrylamide and 11 is a C₁₋₂ alkyl quaternized derivative of the corresponding acrylic acid. Preferred monomer unit weight % concentrations in a homogeneous terpolymer are 60-90% X; 5-30% 1 and 1-30% 11.

The cationic polymer preferably has a weight average molecular weight of 200,000 to 2,000,000, more preferably 300,000 to 1,000,000 and most preferably 400,000 to 800,000 as measured by conventional GPC techniques. The cationic polymer is a water soluble or water dispersible polymer which forms a clear humidity resistant, continuous, hydrophobic film when cast upon a support surface. Further, the cationic polymer is surface active and hydrolytically stable. In the cosmetic compositions of the present invention, a wide variety of enhanced skin and hair altering effects are achieved by the addition of between about 0.01 and about 10%, preferably 0.1 to 10 wt % based on the total weight of the composition of the cationic polymer to a standard or equivalent hair or skin formulation.

As illustrated above, monomer mixtures of X, 1 and 11 including mixtures of amino esters and amino amides and/or intermixtures of quaternized and non-quaternized amino ester and amino amides of 1 and 11 can be employed in the cosmetic formulations of this invention. Thus, the cationic polymer can comprise the described lactam, with an amino ester non-quaternized component and a quaternized amino amide derivative of acrylic acid monomer or with an amino amide non-quaternized component and a quaternized amino ester component in the terpolymer of X, 1 and 11.

In preferred embodiments, the vinyllactam or alkylvinyllactam monomer is a compound of structure (IV): 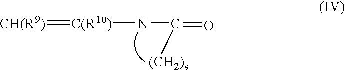
in which:
s denotes a whole number from 3 to 6,
R⁹ and R¹⁰ each denote a hydrogen atom or a C₁-C₅ alkyl radical, provided that at least one of R⁹ and R¹⁰ is H. In a particularly preferred embodiment the vinyllactam is vinylpyrrolidone.

In preferred embodiments, R³, R⁴, R⁵ are independently H or a C₁-C₃₀ linear or branched alkyl radical. In a most preferred embodiment, R₅ contains at least 8 carbon atoms.

The cationic polymer can contain one or more additional monomers, which can be cationic or nonionic, such as, for example, acrylamide, hydroxyethylethyl methacrylate, vinyl imidazole, quaternized vinyl imidazole and the like.

In preferred embodiments, the cationic polymer contains, by weight, (a) 40 to 95% monomer IV, (b) 0.25 to 50% monomer (II), and (c) 0.1 to 55% monomer (I). Further in preferred embodiments, the cationic polymer is a terpolymer chosen from vinylpyrrolidone/dimethylaminopropylmethacrylamide/dodecyldimethylmethacrylamidopropyl-ammonium tosylate terpolymers; vinylpyrrolidone/dimethylaminopropylmethacrylamide/cocoyldimethylmethacrylamidoamidopropylammonium tosylate terpolymers; vinylpyrrolidone/dimethylaminopropylmethacrylamide/lauryldimethylmethacrylamidopropylammonium tosylate or chloride terpolymers.

Any known conditioning agent is useful in the hair compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin altering agent is useful in the compositions of this invention. Preferred conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

Suitable animal and vegetable oils include sunflower, corn, soy, avocado, jojoba, squash, raisin seed, sesame seed, walnut oils, fish oils, glycerol tricaprocaprylate, Purcellin oil or liquid jojoba.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax.

The cationic polymers (other than the poly(alkyl)vinyllactam polymers according to the invention) that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 and 5,000,000 and preferably between 1000 and 3,000,000.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:

(1) homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name GAFQUAT by International Specialty Products; the dimethyl amino ethyl methacrylate/vinyl caprolactam/vinyl pyrrolidone terpolymers, such as the product sold under the name GAFFIX VC 713 by International Specialty Products; the vinyl pyrrolidone/methacrylamidopropyl dimethylamine copolymer, marketed under the name STYLEZE CC 10 by International Specialty Products; and the vinyl pyrrolidone/quaternized dimethyl amino propyl methacrylamide copolymers such as the product sold under the name GAFQUAT HS 100 by International Specialty Products.

(2) derivatives of cellulose ethers containing quaternary ammonium groups, such as hydroxy ethyl cellulose quaternary ammonium that has reacted with an epoxide substituted by a trimethyl ammonium group.

(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.

(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.

(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.

(6) water-soluble polyamino amides prepared by polycondensation of an acid compound with a polyamine. These polyamino amides may be reticulated.

(7) derivatives of polyamino amides resulting from the condensation of polyalcoylene polyamines with polycarboxylic acids followed by alcoylation by bi-functional agents.

(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) the cyclopolymers of alkyl dialyl amine or dialkyl diallyl ammonium such as the homopolymer of dimethyl diallyl ammonium chloride and copolymers of diallyl dimethyl ammonium chloride and acrylamide.

(10) quaternary diammonium polymers such as hexadimethrine chloride. Polymers of this type are described particularly in U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

(11) quaternary polyammonium polymers, including, for example, Mirapol®A 15, Mirapol®AD1, Mirapol®AZ1, and Mirapole 175 products sold by Miranol.

(12) the quaternary polymers of vinyl pyrrolidone and vinyl imidazole such as the products sold under the names Luviquat® FC 905, FC 550, and FC 370 by BASF.

(13) quaternary polyamines.

(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl(C₁-C₂₀) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. Particularly preferred are amino functional silicones.

The silicones may be used in the form of emulsions, nano-emulsions, or micro-emulsions.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C₁-C₁₈ alkyl.

Hydrolyzed proteins include Croquat L, in which the quaternary ammonium groups include a C₁₋₂ alkyl group, Croquat M, in which the quaternary ammonium groups include C₁₀-C₁₈ alkyl groups, Croquat S in which the quaternary ammonium groups include a C₁₋₈ alkyl group and Crotein Q in which the quaternary ammonium groups include at least one C₁-C₁₈ alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

According to the invention, the conditioning agent can be a ceramide type of compound such as a ceramide, a glycoceramide, a pseudoceramide, or a neoceramide. These compounds can be natural or synthetic. Compounds of the ceramide type are, for example, described in Patents pending DE4424530, DE4424533, DE4402929, DE4420736, WO95/23807, WO94/07844, EP-A-0646572, WO95/16665, FR-2 673 179, EP-A-0227994, WO 94/07844, WO 94/24097, and WO 94/10131. Ceramide type compounds useful herein include 2-N-linoleoyl amino-octadecane-1,3-diol, 2-N-oleoyl amino-octadecane-1,3-diol, 2-N-palmitoyl amino-octadecane-1,3-diol, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl) malonamide, N(2-hydroxy ethyl)-N-(3-cetoxyl-2-hydroxy propyl)amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyidimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. Fluorinated oils include perfluoropolyethers described in EP-A-486135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition.

The composition of the invention can contain one or more protecting agents to prevent or limit the degrading effects of natural physical and/or chemical assaults on the keratinous materials.

The protecting agent can be chosen from hydrosoluble, liposoluble and water-insoluble UV filters, antiradical agents, antioxidants, vitamins and pro-vitamins. The above-described cationic polymer enhances the deposition of these materials onto the hair or skin substrate enhancing protection of hair to UV damage.

Organic UV filters (systems that filter out UV rays) can be chosen from among hydrosoluble or liposoluble filters, whether siliconated or nonsiliconated, and mineral oxide particles, the surface of which may be treated.

Hydrosoluble organic UV filters may be chosen from para-amino benzoic acid and its salts, anthranilic acid and its salts, salicylic acid and its salts, hydroxy cinnamic acid and its salts, sulfonic derivatives of benzothiazoles, benzimidizoles, benzoxazoles and their salts, sulfonic derivatives of benzophenone and their salts, sulfonic derivatives of benzylidene camphor and their salts, derivatives of benzylidene camphor substituted by a quaternary amine and their salts, derivatives of phthalydenecamphosulfonic acids and their salts, sulfonic derivatives of benzotriazole, and mixtures thereof.

Hydrophilic polymers which have light-protective qualities against UV rays can be used. These include polymers containing benzylidene camphor and/or benzotriazole groups.

Suitable liposoluble organic UV filters include derivatives of para-aminobenzoic acid, such as the esters or amides of para-aminobenzoic acid; derivatives of salicylic acid; derivatives of benzophenone; derivatives of dibenzoyl methane; derivatives of diphenyl acrylates; derivatives of benzofurans; UV filter polymers containing one or more silico-organic residues; esters of cinnamic acid; derivatives of camphor; derivatives of trianilino-s-triazine; the ethylic ester urocanic acid; benzotriazoles; derivatives of hydroxy phenyl triazine; bis-resorcinol-dialkyl amino triazine; and mixtures thereof.

The liposoluble (or lipophilic) organic UV filter according to the invention can be chosen from octyl salicylate; 4-tert-butyl-4′-methoxy dibenzoyl methane; octocrylene; 4-methoxy cinnamate; 2-ethylhexyl [2-ethylhexyl 4-methoxycinnamate]; and 2-(2H-benzotriazol-2-yl)-4-methyl-6-[2-methyl-3-[1,3,3,3-tetramethyl-1-[(trimethyl silyl)oxy] disiloxanyl]propynyl]phenol.

Other UV filters particularly preferred for use herein are derivatives of benzophenones such as 2-hydroxy-4-methoxy benzophenone-5-sulfonic acid, 2-hydroxy-4-methoxy benzophenone, derivatives of benzalmalonates such as poly dimethyl/methyl(3(4-(2,2-bis-ethoxy carbonyl vinyl)-phenoxy)-propenyl) siloxane, derivatives of benzylidene camphor such as b-b′camphosulfonic [1-4 divinylbenzene] acid and derivatives of benzimidazole such as 2-phenylbenzimidazol-5-sulfonic acid.

Water-insoluble UV filters include various mineral oxides. The mineral oxides may be selected from among titanium oxides, zinc oxides, and cerium oxides. The mineral oxides can be used in the form of ultrafine nanoparticles.

Preferred UV filters include Escalol HP-610 (dimethylpabamido propyl laurdimonium tosylate and propylene glycol stearate) and Crodasorb HP (polyquaternium 59).

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent can be present in an amount 0.001% to 20% by weight, preferably from 0.01% to 10% by weight, and more preferably 0.1 to 5% by weight of the total weight of the final composition.

The composition of the invention can contain a fixing agent.

The fixing agent can be an anionic polymer chosen from polymers containing carboxylic units derived from unsaturated carboxylic mono- or polyacids of the formula: 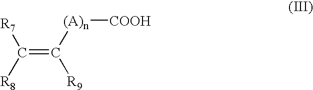
in which n is a whole number from 0 to 10, A₁ denotes a methylene group, optionally bonded to the carbon atom of the unsaturated group or to a neighboring methylene group when n is greater than 1 by means of a heteroatom like oxygen or sulfur, R₇ denotes a hydrogen atom, a phenyl or benzyl group, R₈ denotes a hydrogen atom, a lower alkyl or carboxyl group, R⁹ denotes a hydrogen atom, a lower alkyl group, a —CH₂—COOH, phenyl or benzyl group and polymers containing units derived from sulfonic acid like vinylsulfonic, styrenesulfonic, acrylamidoalkylsulfonic units.

The fixing agent can be an amphoteric polymer chosen from the polymer containing recurring units derived from:
a) at least one monomer chosen from acrylamides or methacrylamides substituted on the nitrogen with an alkyl radical,
b) at least one acid copolymer containing one or more reactive carboxyl groups, and
c) at least one basic comonomer, such as esters with primary, secondary, tertiary, and quaternary amino substituents of acrylic and methacrylic acids and the product of quaternization of dimethylaminoethyl methacrylate with dimethyl or diethyl sulfate.

The fixing agent can be a nonionic polymer chosen from polyalkyloxazolines; vinyl acetate homopolymers; vinyl acetate and acrylic ester copolymers; vinyl acetate and ethylene copolymers; vinyl acetate and maleic ester copolymers; polyethylene and maleic anhydride copolymers; homopolymers of alkyl acrylates; homopolymers of alkyl methacrylates; copolymers of acrylic esters; copolymers of alkyl acrylates and alkyl methacrylates; copolymers of acrylonitrile and a nonionic monomer chosen from among butadiene and alkyl(meth)acrylates; copolymers of alkyl acrylate and urethane; and polyamides.

The fixing polymer can be a functionalized or unfunctionalized, silicone or non-silicone polyurethane.

The fixing polymer can be a polymer of the grafted silicone type containing a polysiloxane portion and a portion consisting of a nonsilicone organic chain, with one of the two portions forming the main chain of the polymer, and with the other being grafted onto said main chain.

The fixing agent can be present in the composition in a relative weight concentration between 0.1 and 10%, preferably 0.5 and 5%.

The composition of the invention can contain an oxidizing agent. The oxidizing agent can be chosen from the group of hydrogen peroxide, urea peroxide, alkali metal bromates, ferricyanides, persalts, and redox enzymes, optionally with their respective donor or cofactor. In a particularly preferred embodiment, the oxidizing agent is hydrogen peroxide. The oxidizing agent can be a solution of oxygenated water whose titer varies from 1 to 40 volumes.

The composition of the invention can contain at least one reducing agent in amounts from 0.01 to 30 wt %, preferably 0.05 to 20 wt % of the total weight of the composition. The reducing agents useful in the practice of this invention can be selected from thiols, like cysteine, thioglycolic acid, thiolactic acid, their salts and esters, cysteamine, and its salts or sulfites. In the case of compositions intended for bleaching, ascorbic acid, its salts and its esters, erythorbic acid, its salts and its esters, and sulfinates, like sodium hydroxymethanesulfinate can be used.

The composition of the invention can contain a dye selected from the group consisting of neutral acid or cationic nitrobenzene dyes, neutral acid or cationic azo dyes, quinone dyes, neutral, acid or cationic anthraquinone dyes, azine dyes, triarylmethane dyes, indoamine dyes and natural dyes. The dye or dyes can be present in a concentration from 0.001 to 20% and preferably 0.005 to 10 wt % based on the total weight of the composition.

The composition of the invention can contain at least one amphoteric polymer or a cationic polymer different from the cationic poly(vinyllactam) defined above. Suitable cationic polymers include a poly(quaternary ammonium) consisting of recurrent units corresponding to the following formulae (W) and (U):

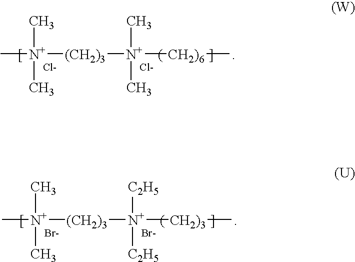

Suitable amphoteric polymers include a copolymer containing at least one acrylic acid and a dimethyldiallyammonium salt as a monomer. The cationic or amphoteric polymer or polymers can be present in an amount of 0.01 to 10%, preferably 0.05 to 5%, and more preferably 0.1 to 3% by weight of the total weight of the composition.

In addition, the compositions according to the invention advantageously include at least one surfactant, which can be present in an amount of 0.1% and 60% preferably 1% and 40%, and more preferably 5% and 30% by weight based on the total weight of the composition. The surfactant may be chosen from among anionic, amphoteric, or non-ionic surfactants, or mixtures of them known to be useful in personal care suitable

The composition of the invention can contain one or more additional cosmetically acceptable additives chosen from conditioning agents, protecting agents, such as, for example, hydrosoluble, liposoluble and water-insoluble UV filters, antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, hydroxy acids, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These additives are present in the composition according to the invention in proportions that may range from 0 to 20% by weight in relation to the total weight of the composition. The precise amount of each additive may be easily determined by an expert in the field according to its nature and its function.

The compositions according to the invention may be used to wash and treat keratinous material such as hair, skin, eyelashes, eyebrows, fingernails, lips, and hairy skin.

The compositions according to the invention can be detergent compositions such as shampoos, bath gels, and bubble baths. In this mode, the compositions will comprise a generally aqueous washing base. The surfactant or surfactants that form the washing base may be chosen alone or in blends, from known anionic, amphoteric, or non-ionic surfactants. The quantity and quality of the washing base must be sufficient to impart a satisfactory foaming and/or detergent value to the final composition. The washing base can be from 4% to 50% by weight, preferably from 6% to 35% by weight, and even more preferentially from 8% to 25% by weight of the total weight of the final composition.

The pH of the composition applied to the keratinous material is generally between 2 and 12. It is preferably between 3 and 8, and may be adjusted to the desired value by means of acidifying or alkalinizing agents that are well-known in the state of the art in compositions applied to keratinous materials. Thus, the composition of the invention can contain at least one alkalizing or acidifying agent in amounts from 0.01 to 30 wt % of the total weight of the composition.

The alkalizing agent can be chosen from ammonia, alkali carbonates, alkanolamines, like mono-, di- and triethanolamines, as well as their derivatives, hydroxyalkylamines and ethoxylated and/or propoxylated ethylenediamines, sodium or potassium hydroxides and compounds of the following formula (XIX): 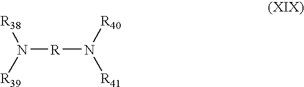
in which R is a propylene residue optionally substituted with an hydroxyl group or a C₁-C₄ alkyl radical; R₃₈, R₃₉, R₄₀ and R₄₁, identical or different, represent a hydrogen atom, a C₁-C₄ alkyl radical or C₁-C₄ hydroxyalkyl radical.

The acidifying agent can be chosen from mineral or organic acids, like hydrochloric acid, orthophosphoric acid, carboxylic acids like tartaric acid, citric acid, or lactic acid, or sulfonic acids and the like.

The physiological and cosmetically acceptable medium may consist exclusively of water, a cosmetically acceptable solvent, or a blend of water and a cosmetically acceptable solvent, such as a lower alcohol composed of C₁ to C₄, such as ethanol, isopropanol, t-butanol, n-butanol, alkylene glycols such as propylene glycol, and glycol ethers. However, the compositions of the invention can be anhydrous.

Generally the present cosmetic compositions are prepared by simple mixing procedures well known in the art.

The invention also has as its object a process for treating keratinous material including the skin or hair, characterized in that it consists of applying to skin or keratinous materials a cosmetic composition as described above, and then eventually rinsing it with water. Accordingly, the process according to the invention makes it possible to maintain the hairstyle, treatment, care, washing, or make-up removal of the skin, the hair, and any other keratinous material.

The compositions according to the invention may also take the form of after-shampoo compositions, to be rinsed off or not, for permanents, straightening, waving, dyeing, or bleaching, or the form of rinse compositions to be applied before or after dyeing, bleaching, permanents, straightening, relaxing, waving or even between the two stages of a permanent or straightening process.

The compositions of the invention may also take the form of skin-washing compositions, and particularly in the form of solutions or gels for the bath or shower, or of make-up removal products.

The compositions of the invention may also be in the form of aqueous or hydro-alcoholic solutions for skin and/or hair care.

The compositions of this invention have demonstrated thermal protection from heat styling tools, more specifically curling or straightening irons. In this application, the cationic polymer provides a continuous film on hair that does not degrade nor whiten upon regular exposure to curling irons at temperatures up to 152° C. Protection is observed via tryptophan measurements and perceived by consumer in combing force reduction.

The compositions described herein are useful in products for personal care, including, but mot limited to, gels, lotions, glazes, glues, mousses, sprays, fixatives, shampoos, conditioners, 2n1 shampoos, temporary hair dyes, semi-permanent hair dyes, permanent hair dyes, straighteners, permanent waves, relaxers, creams, putties, waxes, pomades, moisturizers, mascaras, lip balms and foam enhancers.

The following compositions are representative of hair and skin altering formulations. The ACP 1234 polymer in the following examples is VP/DMAPMA/QDMAPMA in 74/15/11 wt. % monomer concentration.

### EXAMPLE 1

Aerosol Hair Styling Mousse

| Ingredients | Concentration (grams) |
|---|---|
| Deionized water | 93.3 |
| OLETH 20¹ | 0.5 |
| Polysorbitate 20 | 1.0 |
| ACP 1234 | 5.0 |
| SURFADONE LP 100² | 0.1 |
| D-Panthenol³ | 0.1 |

¹Polyethylene glycol ether of oleyl alcohol
²N-octyl pyrrolidone
³Panthothenyl alcohol

A propene/butene propellant was employed to pressurize the above mixture in a metal container to provide a spray.

### EXAMPLE 2

Non-Aerosol Styling Mousse

Concentration (wt. %)

| Ingredients | Formulation A | Formulation B |
|---|---|---|
| Deionized water | 93.15 | 93.35 |
| Cocamidopropyl betaine | 1.0 | 0.75 |
| ACP 1234 | 5.0 | 5.0 |
| OLETH 20 | 0.5 | 0.25 |
| DL-Panthenol | 0.1 | 0.1 |
| Germall Plus⁴ preservative | 0.25 | 0.25 |
| pH | 5.2 | 5 |

⁴Imidazolidinyl urea 99% + iodopropynyl butyl carbamate (IPBC)1%

### EXAMPLE 3

Hair Texture Improving Non-Aerosol Styling Mousse

| Ingredients | Concentration (wt. %) |
|---|---|
| Deionized water | 95.15 |
| PQ-4 (CELQUAT 1-200⁵) | 0.5 |
| GAFQUAT PQ-11⁶ | 2.0 |
| Cocamidopropyl betaine | 1.0 |
| ACP 1234 | 0.5 |
| OLETH 20 | 0.5 |
| DL-Panthenol | 0.1 |
| Germall Plus | 0.25 |
| pH | 6.4 |

⁵Dimethyl diallyl ammonium chloride grafted on hydroxy ethyl cellulose
⁶Quaternized vinyl pyrrolidone/dimethylaminoethyl methacrylate copolymer

### EXAMPLE 4

Aerosol Styling Mousse Concentrate

Concentration (wt. %)

| Ingredients | Formulation A | Formulation B |
|---|---|---|
| Deionized water | 83.2 | 83.7 |
| ACP 1234 | 5.0 | 5.0 |
| Polysorbate 29 | 1.0 | 0.75 |
| OLETH 20 | 0.5 | 0.25 |
| SURFADONE LP-100 | 0.1 | 0.1 |
| DL-Panthenol | 0.1 | 0.1 |
| Germall Plus | 0.1 | 0.1 |
| Propellant | | |
| HF 152a⁷ | 4.75 | 4.75 |
| A-17⁸ | 4.75 | 4.75 |
| pH | 4.4 | 5.25 |

⁷Fluorinated propellant
⁸Hydrocarbon propellant

After thorough mixing the above mixtures were poured into separate cans and pressurized with propellant. These mousse formulations were tested over a period of 3 months for stability and found to show no darkening or reduction in resistance to humidity.

The hair treated with formulations of Example 4 styling mousse exhibited high shine, curl snap and curl memory.

### EXAMPLE 5

Hair Sculpting Lotion

| Ingredients | Concentration (wt. %) |
|---|---|
| Deionized water | 93.85 |
| OLETH 20 | 0.5 |
| ACP 1234 | 5.0 |
| DL-Panthenol | 0.1 |
| Methyl gluceth-10⁹ | 0.2 |
| Silk amino acids | 0.1 |
| Hydrolyzed wheat protein | 0.1 |
| Germall Plus | 0.15 |

⁹Ethoxylated methylglucose

### EXAMPLE 6

Hair Mask

| Ingredients | Concentration (wt. %) |
|---|---|
| Part A | |
| Deionized water | 67.1 |
| Disodium | 0.1 |
| Stabileze | 0.2 |
| Part B | |
| Prolipid 151¹⁰ | 3.0 |
| Ceraphyl 791/494¹¹ | 3.0 |
| Mineral oil | 3.0 |
| Avacado oil | 1.0 |
| Jojoba¹² | 1.0 |
| Cerasynt¹³ M/D | 1.5 |
| Part C | |
| Water | 3.0 |
| ACP 1234 | 93.46 |
| Methyl gluceth 20 | 0.5 |
| Parathenol | 0.5 |
| Lubrajel¹⁴ CG | 3.0 |
| Liquapar Optima (preservative) | 0.5 |

¹⁰Glyceryl stearate (and) cetyl alcohol (and) stearyl alcohol (and) behenyl alcohol (and) palmitic acid (and) stearic acid (and) hydroxyethyl cetearamidopropyldimonium chloride
¹¹Isocetyl stearoyl stearate
¹²Wax
¹³Stearamide MEA stearate
¹⁴Glyceryl polymethacrylate and propylene glycol

Parts A and B were separately prepared and then mixed in a high speed mixer for about 18 minutes, after which part C was added and homogenized for an additional 18 minutes and then cooled to about 45° C. Mixing of the resulting cooled homogenized mixture was continued at moderate speed for 10 minutes before adding and mixing in Part D and Liquapar Optima. The homogenized product had a pH of 5.06 and a Brookfield viscosity of 4.6×10³ Cps using a RVT DV-11 Brookfield viscometer TB at 20 rpm.

The mousse is applied to the hair for 15 minutes before rinsing off. The hair when dried has a soft shimmering texture. Styling produces a set with superior curl snap and curl memory.

### EXAMPLE 7

Aerosol Mousse-Foam Thickness

| | Formulations | | | | |
|---|---|---|---|---|---|
| | A | B | C | D | E |
| Ingredients | Concentration (wt. %) | | | | |
| Deionized water | 76.8 | 79.35 | 74.35 | 81.95 | 80.55 |
| OLETH 20 | 1.0 | 1.0 | 1.0 | — | 1.0 |
| Polysorbate 20 | 1.0 | 1.0 | 1.0 | — | — |
| Panthenol 50% | 0.1 | 0.1 | 0.1 | — | 0.1 |
| Dimethicone copolyol | 0.1 | 0.1 | 0.1 | — | 0.1 |
| Ceraphyl 41¹⁵ | 0.3 | 0.3 | 0.3 | — | 0.1 |
| Tocopheryl acetate | 0.1 | 0.1 | 0.1 | — | 0.1 |
| Germall Plus | 0.5 | 0.5 | 0.5 | 0.5 | 0.5 |
| ACP 1234 | 10.1 | 5.05 | 10.1 | 5.05 | 5.05 |
| Gafquat 755N | — | 2.5 | 2.5 | 2.5 | 2.5 |
| Foam Density | Very high | High | Very high | High | High |

¹⁵C₁₂ to C₁₅ alkyl lactate

### EXAMPLE 8

### Conditioning Cream Rinse Formulation

Part A

86.4% Deionized H₂O
0.5% SLES (Cerasynt LP; Glycol stearate/Nalaureth sulfate/hexyl glycol available from ISP)
0.1% NaEDTA (Ethylenediaminetetraacetic acid disodium salt dihydrate; Aldrich)
   Part B
2.55 Cetyl Stearyl Alcohol (Lanette Wax 0; Henkel Corporation)
   Part C
10% Aculyn 46 (modified polyethylene glycol, enzymatically modified starch; Rohm & Haas)
   Part D
0.5% Conditioning terpolymer ACP 1234
   Instructions
   Heat Part A to 60° C. with moderately slow stirring. Add Part B to Part A once
   Part A appears well mixed and homogeneous. Continue slow stirring and allow solution to cool to an ambient temperature. Add Part C while stirring and eventually add Part D.

### EXAMPLE 9

### Conditioning Shampoo Formulation

Part A

15% Ammonium Lauryl Sulfate (Standapol A, Henkel Corporation)
15% Sodium Lauryl Sulfate (Rhodapon SB-8208/S, Rhône Poulenc)
8% Cocamidopropyl Betaine (Mitratine CB, Rhône Poulenc)
2% Lauramide DEA (Monamid 716, Mona Industries)
   Part B
1% Conditioning terpolymer of Example 6
58.8% Deionized H₂O
   Part C
0.2% Diazolidinyl Urea/IPBC (Germall Plus, ISP)
   Instructions
   Heat Part A to 60° C. with moderately slow stirring for approximately ½ hr. or until solution becomes transparent. At the same time, heat Part B to 55° C. while stirring until homogeneous solution is obtained. Add Part B to Part A while continuously stirring. Remove temperature source. Once the resulting solution has reached 45° C., add Part C. Continue to stir (slowly) until the target solution has cooled to an ambient temperature.

In the conditioners and shampoo formulations tested under actual use conditions in comparison with similar formulations with known polymers, the terpolymers of the invention have excellent wet combing, excellent dry feel and softness, and excellent wet feel.

### EXAMPLE 10

### Styling Lotion

A styling lotion formulation was prepared in aqueous solution using 1% by weight of the terpolymer ACP 1234 and 0.1% preservative. This lotion had excellent hold and resistance to humidity.

### EXAMPLE 11

### Styling Mousse

A concentrate is prepared by dissolving 1 g of the terpolymer of Example 1, 0.1 g of preservative and 98.9 g of deionized water.

A styling mousse formation is prepared by mixing 70 g of the concentrate and 30 g of hydrocarbon propellant in an aerosol can provided with a mousse dispenser.

This example is repeated using VP/DMAPMA/DMAPMA quaternized with hexadecyl bromide which provides excellent heat protecting properties to the hair.

### EXAMPLE 12

The following aqueous bleaching ready-for-use composition is prepared (amounts expressed in grams):

| | |
|---|---|
| Hydrogen peroxide to 200 volumes | 12 |
| Stabilizer | qs |
| ACP 1234 A** | 5.0 AS* |
| pH agent, qs | pH 4.7 |
| Water, qsp | 100 |

*AS = active substance
**Vinylpyrrolidone/dimethylaminopropylmethacrylamide/lauryldimethylmethacrylamidoammonium tosylate and chloride mixture

The above bleaching composition was applied and left for 45 minutes beneath a hood to natural hair, then rinsed abundantly with water. Brightening of the hair was obtained.

### EXAMPLE 13

The following bleaching composition is prepared (amounts expressed in grams):

Anhydrous composition

| | |
|---|---|
| Potassium persulfate | 35 |
| Sodium persulfate | 30 |
| Sodium metasilicate | 14 |
| Ammonium chloride | 5 |
| EDTA | 1 |
| Sodium dioctylsulfosuccinate/ | 1 |
| sodium benzoate | |
| Calcium stearate | 1 |
| ACP 1234 | 3 AS* |
| Silica | 7 |

40 g of the above anhydrous composition is mixed with
80 g of the following aqueous composition:

Aqueous composition

| | |
|---|---|
| Cetearyl alcohol/ceteareth 30 | 2.85 |
| Stabilizers | 0.06 |
| Sequestering agent | 0.15 |
| Hydrogen peroxide to | 9 |
| 200 volumes | |
| Phosphoric acid, qs | pH 2 |
| Distilled water, qsp | 100 |

*AS = active substance

A bleaching cream ready-for-use is obtained, which applied and left for 45 minutes under a hood, permitted strong and homogeneous bleaching of dark natural hair to be obtained.

### EXAMPLE 14

Permanent Wave Composition:

The following reducing composition is prepared (amounts expressed in grams):

| | |
|---|---|
| VP/DMAPMA/QDMAPMA (50/39/11) | 2.0 |
| Thioglycolic acid | 9.2 |
| Arginine | 15 |
| 20% ammonia NH₃ | 9.3 |
| Ammonium carbonate | 4.5 |
| Cocoylamidopropylbetaine/glycerol | 1.3 |
| monolaurate (25/5) in 30% aqueous solution | |
| Peptizing agent | 0.8 |
| Isostearyl alcohol (Tego Alkanol 66 | 12 |
| sold by Goldschmidt) | |
| Sequestering agent | 0.4 |
| Perfume | 0.4 |
| Demineralized water, qsp | 100 |

This reducing composition is applied to a lock of moist hair wound onto a curler beforehand 9 mm in diameter. After 10 minutes of waiting it is rinsed abundantly with water. The following oxidizing composition is then applied:

Oxidizing composition:

| | | |
|---|---|---|
| Hydrogen peroxide (35%) | 10 | g |
| Tetrasodium pyrophosphate (0.02 g) and | | |
| sodium stannate (0.04 g) | | |
| Sequestering agent: pentasodium pentaacetate | 0.06 | AS* |
| Demineralized water, qsp | 100 | g |

*AS denotes active substance

After 10 minutes of waiting, the lock is abundantly rinsed again. The hair is then unwound from the curler and dried. The lock is waved.

### EXAMPLES 15-17

The following hair fixative compositions are in accordance with this invention.

### EXAMPLE 15

Foam Without Propellant (Pump Bottle Airspray F2)

| | |
|---|---|
| Quat. Terpolymer*** | 2.5% |
| PVP/VA | 1% |
| Glycerol | 0.5% |
| Preservatives, perfume | |
| Water | qsp |
| | 100% |

### EXAMPLE 16

Aerosol Foam

| | |
|---|---|
| Quat. Terpolymer*** | 2.5% |
| PVP | 1% |
| Aerogaz 3.2 N (Atochem) | 6% |
| Preservatives, perfume | |
| Water | qsp |
| | 100% |

### EXAMPLE 17

Gel

| | |
|---|---|
| Quat. Terpolymer*** | 2.5% |
| PVP/VA | 0.5% |
| Jaguar HP 105 | 1% |
| Preservatives, perfume | |
| Water | qsp |
| | 100% |

***Vinylpyrrolidone/dimethylaminopropyl-methacrylamide/lauryldimethylmethacrylamidoammonium chloride terpolymer (74/15/11) prepared according to U.S. Patent 6,207,778.

### EXAMPLE 18

The following aqueous bleaching ready-for-use composition was prepared (amounts expressed in grams):

| | |
|---|---|
| Citric acid | 7.4 |
| Dihydrated trisodium citrate | 1 |
| Hydroxyethylcellulose | 1.5 |
| 2-Oxoglutaric acid | 0.8 |
| Sodium ascorbate | 5.7 |
| L-cysteine | 2 |
| VP/DMAPMA/QDMAPMA** | 0.3 AS* |
| Magnesium sulfate | 1 |
| Water, qsp | 100 |

*AS = active substance
**Vinylpyrrolidone/dimethylaminopropylmethacrylamide/lauryldimethylmethacrylamidoammonium chloride terpolymer (72/16/12) prepared according to U.S. Patent 6,207,778.

The above bleaching composition permitted regular bleaching of artificially dyed hair with an oxidation dye.

### EXAMPLE 19

The following permanent wave composition was prepared (amounts expressed in grams):

| | | |
|---|---|---|
| Thioglycolic acid | 9.2 | |
| 20% ammonia NH₃ | 9.3 | |
| Ammonium carbonate | 4.5 | |
| Cocoylamidopropylbetaine/glycerol | 0.4 | AS* |
| monolaurate (25/5) | | |
| EDTA | 0.4 | |
| Cationic polymer of formula W | 1 | AS* |
| in 60% solution in water | | |
| VP/DMAPMA/QDMAPMA** | 0.3 | AS* |
| Water, qsp | 100 | |

*AS = active substance
**Vinylpyrrolidone/dimethylaminopropylmethacrylamide/lauryldimethyl methacrylamidoammonium chloride terpolymer (74/15/11).

The above permanent wave composition was applied for 15 minutes to wetted hair, wound beforehand onto rollers into curls, then rinsed abundantly with water. A solution of oxygenated water of 8 volumes and pH 3 was then applied for 5 minutes, then rinsed again and the rollers removed and the hair dried.

The hair presented an excellent homogeneous hair style.

### EXAMPLE 20

The following aqueous bleaching ready-for-use composition was prepared (amounts expressed in grams):

| | |
|---|---|
| VP/DMAPMA/QDMAPMA** | 1 |
| Isostearyl alcohol (Tego Alkanol 66 | 12 |
| sold by Goldschmidt) | |
| Benzyl alcohol | 10 |
| Sodium hydroxymethanesulfonate | 7 |
| Adjusted to pH | 3 |
| Demineralized water, qsp | 100 |

**Vinylpyrrolidone/dimethylaminopropylmethacrylamide/lauryldimethyl methacrylamidoammonium chloride terpolymer (74/15/11).

The above bleaching composition was applied to natural gray hair with 90% white, dyed beforehand with shade 20 (copper) of the L'Oreal Movida scale with a bath ratio of 10 g per 1 g of hair.

After 30 minutes of waiting, the locks were rinsed and dried.

The copper reflection disappeared, revealing the hair as it was before application of the dye again.

### EXAMPLE 21

The following reducing composition was prepared (expressed in grams):

| | |
|---|---|
| Thioglycolic acid | 9.2 |
| Arginine | 15 |
| 20% ammonia NH₃ | 9.3 |
| Ammonium carbonate | 4.5 |
| Cocoylamidopropylbetaine/glycerol monolaurate | 1.3 |
| (25/5) in a 30% aqueous solution | |
| Peptizing agent | 0.8 |
| ACP 1234 | 1 |
| Oleic alcohol | 12 |
| Sequestering agent | 0.4 |
| Perfume | 0.4 |
| Demineralized water, qsp | 100 |

This reducing composition was applied to a lock of wet hair first wound onto curlers 9 mm in diameter. After 10 minutes of waiting, it was rinsed abundantly with water. An oxidizing composition was then applied (oxygenated water 8 volumes at pH 3). After 10 minutes of waiting, the lock was rinsed again abundantly. The hair was then unwound from the curler and dried. The lock was wavy.

### EXAMPLE 22

The following compositions are prepared (expressed in grams):

| Oxidizing composition: | |
|---|---|
| Cetyl alcohol | 2.3 |
| Ethoxylated aliphatic alcohol | 0.6 |
| Lauramide MEA | 0.9 |
| Glycerol | 0.5 |
| Hydrogen peroxide | 7.5 |
| Perfume | qs |
| Demineralized water, qsp | 100 |
| Dye composition: | |
| Ethoxylated aliphatic alcohols | 32.5 |
| Oleic acid | 2 |
| Oleic alcohol | 1.8 |
| Aliphatic amide | 4 |
| Glycerol | 3 |
| Cationic polymer of formula (W) | 2 |
| in a 60% solution with water | |
| Amphoteric polymer (Merquat 280) | 2 |
| Na EDTA | qs |
| Reducing agent | qs |
| Ammonia (20% NH₃) | 8 |
| 1,4-Diamino-2-nitrobenzene | 0.6 |
| ACP 1234 | 0.3 AS* |
| Water, qsp | 100 |

*AS = active substance

The dye composition was mixed at the moment of use in a plastic bowl and over 2 minutes with the oxidizing composition mentioned above in an amount of 1 part dye composition per 1.5 parts oxidizing composition.

The obtained mixture is applied to locks of natural hair consisting of 90% white hair and allowed to stand for 30 minutes.

The locks are rinsed with water, washed with standard shampoo and rinsed again with water, then dried and untangled.

The hair was dyed to a strong brown shade.

### EXAMPLE 23

Shampoo

| | Concentration (grams) | |
|---|---|---|
| Ingredients | Formulation A | Formulation B |
| Ethoxylated Na laurylether sulfate | 17 | 10 |
| (2 moles ethoxylate) | | |
| Lauric acid | 0.6 | — |
| Cocoyl betaine | 2.5 | 2.5 |
| ACP 1234 | 1.0 | 0.5 |
| Polydimethyl siloxane (Silicone AK) | — | 0.5 |
| Hydroxyethyl cellulose | — | 1.0 |
| Lactic acid to pH 7 | — | |
| DI Water | 100 | 100 |

### EXAMPLE 24

Hand and Body Lotion

| Ingredients | Wt. % |
|---|---|
| Part A | |
| Deionized water | 52.0 |
| Carbomer 1342¹⁶ | 0.4 |
| Glycerin | 3.5 |
| Sorbitol | 2.0 |
| Dimethicone | 10 |
| Mineral oil | 2.0 |
| Part B | |
| Triethanolamine | 0.3 |
| Part C | |
| Deionized water | 25.0 |
| ACP 1234 | 5.5 |
| Fragrance | qs |

¹⁶Crosslinked polymer of acrylic acid

Carbomer is homogenized in a glass vessel with deionized water after which the remaining components of Part A are added in the order listed. The resulting liquid is neutralized with 99% triethanol amine of Part B and components of Part C are then added and mixed until a homogeneous lotion is obtained. This lotion when applied to the skin, exhibits moisturizing and anti-aging properties.

### EXAMPLE 25

Non-Aerosol Hair Styling Mousse with Photo
Color Protectant Dispersible Concentrate

| Ingredients | Amount(g) | % w/w |
|---|---|---|
| ACP 1234 | 5.0 | 5 |
| Photo/color Protectant | 4.0 | 4 |
| Concentrate Composition* | | |
| Oleth-20 | 0.5 | 0.5 |
| Cocamidopropyl betaine | 1.0 | 1.0 |
| D-Panthenol | 0.1 | 0.1 |
| Germall Plus | 0.25 | 0.25 |
| Citric acid to adjust pH to | 5.5 | |
| Distilled water | 89.15 | 89.15 |

*100 g. benzophenone-4 + 50 g. 6M NaOH + 49.5 g. distilled water were mixed until dissolution of the photoabsorber after which 100 g GAFQUAT 755N and 100 g ZnO were added and then milled to a paste.

In the above formulation, prior to the addition of the color protectant, the tertiary polymer, water, surfactants, preservative and Panthenol were stirred until a uniform mixture was obtained. The resulting liquid was then mixed to form a liquid dispersion.

### EXAMPLE 26

Hair Sculpting Lotion Containing Photo/Color
Protectant Dispersible Concentrate

| Ingredients | Amount(g) | % w/w |
|---|---|---|
| ACP 1234 | 5.0 | 5.0 |
| Color protect. of Example 25 | 4.0 | 4.0 |
| Oleth-20 | 0.5 | 0.5 |
| D-Panthenol | 0.1 | 0.1 |
| Methyl Gluceth-10 | 0.2 | 0.2 |
| Keratin amino acids | 0.1 | 0.1 |
| Hydrolyzed wheat protein | 0.1 | 0.1 |
| Germall Plus | 0.15 | 0.15 |
| Distilled water | 89.85 | 89.85 |

The above formulation was prepared in a manner similar to Example 24.

### EXAMPLE 27

Hair Styling Lotion with Photo/Color
Protectant Dispersible Concentrate

| Ingredients | Amount(g) | % w/w |
|---|---|---|
| Phase A | | |
| Hydroxyethyl cellulose | 1.0 | 1.0 |
| Oleth-20 | 0.1 | 0.1 |
| Deionized water | 48.2 | 48.2 |
| Phase B | | |
| Acp 1234 | 5.0 | 5.0 |
| Color Protect. of Example 25 | 4.0 | 4.0 |
| Polysorbate-20 | 1.0 | 1.0 |
| Germall Plus | 0.2 | 0.2 |
| Distilled water | 40.5 | 40.5 |

In the above formulation, Phases A and B were separately compounded before combining in a uniform dispersible mixture.

### EXAMPLE 28

The hair styling lotion of Example 27 was repeated except that, in Phase B, only 2.5 grams of terpolymer and 43 grams of water were employed. Both formulations provided easily dispersible liquids upon shaking and exhibited excellent holding power when applied to the hair.

### EXAMPLE 29

Surfactant Free Aerosol Styling Mousse
with Photo/Color Protectant

| Concentrate Ingredients | Amount(g) | % w/w |
|---|---|---|
| ACP 1234 | 3.75 | 3.75 |
| Color Protect. of Example 25 | 4.0 | 4.0 |
| Germall Plus | 0.05 | 0.05 |
| Distilled water | 92.2 | 92.2 |

### EXAMPLE 30

The styling mousse of Example 29 was repeated except that only 2.5 grams of the terpolymer and 93.45 grams of distilled water were employed. Both formulations were loaded into aerosol containers and both produced a hair fixative having excellent hold.

### EXAMPLE 31

Aerosol Styling Mousse with Photo/Color Protectant

| Concentrate Ingredients | Amount(g) | % w/w |
|---|---|---|
| ACP 1234 | 3.75 | 3.75 |
| Color Protect. of Example 25 | 4.0 | 4.0 |
| Oleth-20 | 0.5 | 0.5 |
| Polysorbate 20 | 1.0 | 1.0 |
| SURFADONE LP-100 | 0.1 | 0.1 |
| Germall Plus | 0.05 | 0.05 |
| Distilled water | 90.5 | 90.5 |

The above ingredients were stirred until a uniform mixture was obtained and the loaded into an aerosol container.

### EXAMPLE 32

The formulation of Example 31 was repeated except that only 2.5 grams of the terpolymer and 91.75 grams of distilled water were employed. Both formulations exhibited excellent hold when applied to hair.

### EXAMPLE 33

Hair Coloring Gel

| Ingredients | wt. % |
|---|---|
| ACP 1234 | 2.5 |
| Decyl glucoside | 5.0 |
| Na Bisulfite | 0.3 |
| L-Ascorbic acid | 0.3 |
| Na EDTA | 0.1 |
| Deionized water | 66.1 |
| C₁₄₋₁₅ Pareth-10 | 10.0 |
| C₁₂₋₁₅ Pareth-3 | 10.0 |
| NH₄ OH | 4.2 |
| Ethanolamine | 3.6 |
| Dyes: | 1.4 |
| 0.35 wt. % p-phenylenediamine | |
| 0.35 wt. % 2-methylresorcinol | |
| 0.25 wt. % resorcinol | |
| 0.25 wt. % p-aminophenol | |
| 0.1 wt. % 4-amino-2-hydroxy toluene | |
| 0.05 wt. % naphthol | |
| 0.05 wt. % N,N-bis(2-hydroxyethyl)-p- | |
| (phenylenediamine sulfate) | |

The above formulation was mixed to uniform composition and an anionic hair dye developer was added, i.e. 3 wt % H₂O₂+1.5 wt. % acrylate/Steareth-20 methacrylate copolymer+0.1 wt. % Na EDTA+95.4 wt. % deionized water. Equal volumes of dye colorant gel and dye developer and mixed before application to the hair. When applied to the hair, the above formulation produced a rich brown color over a natural moussy brown.

### EXAMPLE 34

The coloring gel of Example 33 was repeated except that a non-ionic dye developer was employed, i.e. 3 wt. % H₂O₂+1.5 wt. % PEG-150/stearyl/SMDI copolymer+0.1 wt. % Na EDTA+0.1 wt. % phosphoric acid. This formulation also produced a rich brown color.

### EXAMPLE 35

The following conditioning waving gel is prepared:

| Ingredients | Amount (g) | % w/w |
|---|---|---|
| Phase A | | |
| Ammounium thiglycolate 60% | 15 | 15 |
| Ammoniumn hydroxide 28% | 2 | 2 |
| Monoethanolamine 99% | 4 | 4 |
| EDTA | 0.20 | 0.20 |
| Distilled Water | 21.3 | 21.3 |
| Phase B | | |
| Hydroxyethylcellulose | 1.0 | 1 |
| ACP 1234 | 2.5 | 2.5 |
| Propylene glycol | 4.0 | 4 |
| Distilled Water | 50.0 | 50 |
| Total | 100.0 | 100 |

Phase B is prepared by slowly adding hydroxyethylcellulose and ACP 1234 to propylene glycol in water. Phase A is prepared separately by mixing ingredients. Then Phase A is added to Phase B and mixed until a uniform, clear gel is formed.

### EXAMPLE 36

The following conditioning no-lye hair relaxer was prepared:

| Ingredients | Amount (g) | % w/w |
|---|---|---|
| Part I | | |
| Prolipid 151 | 10.0 | 10.0 |
| Cetearyl Alcohol | 6.0 | 6.0 |
| PEG-75 lanolin | 2.0 | 2.0 |
| White petroleum jelly | 8.0 | 8.0 |
| Light mineral oil | 10.0 | 10.0 |
| Propylene glycol | 2.0 | 2.0 |
| Calcium hydroxide | 5.0 | 5.0 |
| ACP 1234 | 2.5 | 2.5 |
| Deionized water | 54.5 | 54.5 |
| Total | 100 | 100 |
| Part II (Activator) | | |
| Guanidine carbonate | 25.0 | 25.0 |
| Xanthan gum | 0.2 | 0.2 |
| Distilled Water | 74.8 | 74.8 |
| Total | 100.0 | 100.0 |

Part I: heat oil phase ingredients to 60° C. and water to the same temperature. Dissolve ACP 1234 in the water phase. Add water to oils while stirring vigorously. Continue stirring until cream start to set at about 45° C. Add calcium hydroxide and stir to disperse.

Part II: dissolve xanthan gum in the water with stirring and heat to 70° C. Cool to 45° C. and add the guanidine carbonate.

Part I and Part II have to be combined prior to hair application.

### EXAMPLE 37

The following water-proof sunscreen formulation is prepared:

| Ingredients | Amount (g) | % w/w |
|---|---|---|
| Phase A | | |
| ACP 1234 | 2.5 | 2.5 |
| DI water | 35.0 | 35.0 |
| Phase B | | |
| Glycerin | 10.0 | 10.0 |
| PVP (PVP K-30) | 2.0 | 2.0 |
| DI water | 20.0 | 20.0 |
| Phase C | | |
| Stearic acid | 1.80 | 1.80 |
| Tricontanyl PVP | 4.0 | 4.0 |
| Lauryl pyrrolidone | 1.0 | 1.0 |
| Petrolatum | 5.0 | 5.0 |
| Mineral oil | 4.0 | 4.0 |
| Dimethicone | 0.50 | 0.50 |
| Glyceryl stearate | 1.50 | 1.50 |
| Phase D | | |
| Titanium dioxide, 40% | 12.50 | 12.50 |
| Capric/caprylic trigliceride soln | | |
| Isopropylparaben (and) | 0.20 | 0.20 |
| Isobutylparaben (and) butylparaben | | |
| Fragrance | qs | qs |
| Total | 100 | 100 |

Prepare Phase A by dissolving polymer in water. Heat to 80° C. Prepare B by dissolving PVP in water and glycerin. Add B to A with stirring. Mix C with heating to 80° C. When waxes are melted add C to AB. Stir until uniform. Cool to 35° C. Add TiO₂ with stirring, followed by preservative and fragrance (SPF 12).

### EXAMPLE 38

The following body shampoo composition is prepared:

| Ingredients | Amount (g) | % w/w |
|---|---|---|
| Phase A | | |
| Sodium laureth-2 sulfate (28%) | 60.0 | 60.0 |
| Lauramide DEA | 5.0 | 5.0 |
| Lauramide MEA | 5.0 | 5.0 |
| Methyl gluceth-1 | 3.0 | 3.0 |
| PEG-75 lanolin | 4.0 | 4.0 |
| Phase B | | |
| Citric acid (50% aqueous) | 1.5 | 1.5 |
| DI water | 16.5 | 16.5 |
| Phase C | | |
| ACP 1234 | 5.0 | 5.0 |
| Preservative | qs | qs |
| Total | 100 | 100 |

Mix A at room temperature, add B with stirring, add C with stirring at 50° C. When clear and uniform, cool with mixing to room temperature.

### EXAMPLE 39

Moisturizing Body Wash

| | |
|---|---|
| Ingredients | % w/w |

Phase A

| | |
|---|---|
| Deionized water | 7.25 |
| Sodium Laureth Sulfate (Standapol ES-3) | 30.00 |
| Cocamidopropyl Betaine (Rtego Betaine F) | 15.00 |
| PEG-6 Caprylic/Capric Glyceride (Softigen 767) | 6.00 |
| Glycerin | 3.00 |
| Citric Acid | 0.10 |
| Titanium Dioxide (Titanium Dioxide 3328) | 0.50 |
| Triethanolamine, 99% | 1.80 |
| Lauryl Glucoside (and) Sodium Laureth Sulfate | 13.50 |
| (Plantaren PS 200) | |
| Disodium EDTA (Versene NA) | 0.10 |

Phase B

| | |
|---|---|
| Deionized water | 5.50 |
| ACP 1234 | 2.50 |

Phase C

| | |
|---|---|
| Distilled Palm Fatty Acid (Prifac 7971) | 4.50 |
| C₁₂₋₁₅ Alkyl Lactate (CERAPHYL ® 41) | 4.50 |
| Soybean Oil | 4.00 |

Phase D

| | |
|---|---|
| Fragrance (Belmay # 4019-10156) | 0.75 |
| Propylene Glycol (and) Diazolidinyl Urea (and) | 1.00 |
| Methylparaben (and) Propylparaben (GERMABEN ® II) | |
| Total | 100.00% |

Procedure:
1. Combine Phase A ingredients, with mixing between additions; heat to 70-75° C.
2. Combine Phase B ingredients and heat to 55° C.
3. Combine Phase C ingredients and heat to 75° C.
4. Add Phase B to Phase A with mixing when Phase A is at 75° C.
5. Add Phase C to the batch with mixing until batch is uniform. Cool to 40° C.
6. Add Phase D at 40° C. with mixing. QS for water loss and mix to RT.

pH=6.54 Viscosity=24,800 cps (Brookfield Model RVT, TB @ 5 rpm) EXAMPLE 40

Moisturizing Lotion

| | |
|---|---|
| Ingredients | % w/w |

Phase A

| | |
|---|---|
| Deionized water | 67.00 |
| ACP 1234 | 3.00 |
| Disodium EDTA (Versene NA) | 0.10 |
| Glyceryl Polymethacrylate (and) Propylene glycol (and) | 1.00 |
| PVM/MA Copolymer (LUBRAJEL ® Oil) | |
| Glycerin | 1.50 |
| Guar Gum (Jaguar C) | 0.80 |

Phase B

| | |
|---|---|
| Diisopropyl Adipate (CERAPHYL ® 230) | 4.00 |
| Isocetyl Stearate (CERAPHYL ® 494) | 6.00 |
| Ethylhexyl Palmitate (CERAPHYL ® 368) | 10.00 |
| Glyceryl Stearate (and) Cetyl Alcohol (and) Stearyl | 4.00 |
| Alcohol (and) Behenyl Alcohol (and) Palmitic Acid | |
| (and) Stearic Acid (and) Hydroxyethyl | |
| Cetearamidoopropyldimonium Chloride (PROLIPID ® 151) | |

Phase C

| | |
|---|---|
| Cyclopentasiloxane (Si-Tec ™ CM 040) | 2.00 |

Phase D

| | |
|---|---|
| Propylene glycol (and) Diazolidinyl Urea (and) Iodopropynyl | 0.60 |
| Butylcarbamate (LIQUID GERMALL ® PLUS) | |
| Total | 100.00% |

Procedure:
1. Add ACP 1234 to water; mix well. Add Disodium EDTA. Mix well between additions. Pre-mix gum with glycerin and slowly add to Phase A with stirring. Begin heating Phase A to 70-75° C. Add Lubrajel Oil when the gum is well dispersed.
2. Combine Phase B; heat to 75-80° C.
3. When Phase A is stirring at 70-75° C. and Phase B is uniform at 75-80° C., add Phase B to Phase A with homogenization. When batch appears uniform, turn off heat and switch to sweep agitation.
4. Add Phases C and D at 35-40° C.
5. Make up for water loss and sweep to RT.

pH=5.16 Viscosity=36,000 cps (Brookfield Model RVT TB @ 5 rpm) EXAMPLE 41

Mascara

| | |
|---|---|
| Ingredients | % w/w |

Phase A

| | |
|---|---|
| Deionized water | 46.30 |
| Butylene Glycol | 4.00 |
| Disodium EDTA | 0.10 |

Phase B

| | |
|---|---|
| Hydroxyethylcellulose (Natrosol 250 HHR) | 1.00 |

Phase C

| | |
|---|---|
| Kaolin (Kaolin 2457) | 0.50 |
| Nylon-12 (SP-500) | 1.00 |
| Iron Oxides (C33-134 Iron Oxide Black) | 10.00 |

Phase D

| | |
|---|---|
| Beeswax | 10.00 |
| Copernicia Cerifera (Carnauba) Wax (Carnauba Wax #1 | 6.60 |
| Yellow Bleached) | |
| PEG-20 Stearate (CERASYNT ® 840) | 2.00 |
| Glyceryl Stearate (and) Laureth-23 (CERASYNT ® 945) | 3.00 |

Phase E

| | |
|---|---|
| ACP-1234 | 10.00 |
| Alcohol SD 40B | 5.00 |

Phase F

| | |
|---|---|
| Sodium Hydroxymethylglycinate (SUTTOCIDE ® A) | 0.50 |
| Total | 100.00% |

Procedure:
1. Heat water to 82-85° C.; add remaining Phase A ingredients and mix until uniform.
2. Sprinkle Natrosol into batch using slow homo-mixing. Avoid aeration.
3. Add Phase C color phase (pre-pulverized) to main batch. Mix until uniform using homo-mixing.
4. Heat Phase D to 87° C.
5. Add Phase D to main batch using homo-mixing. Mix until uniform.
6. Switch to sweep-mixing; cool batch to 50° C. Add pre-mixed Phase E, mix until uniform.
7. At 45° C. add Phase F; mix until uniform.
8. Continue sweep-mixing and slow cooling to 30° C. Fill containers.
   pH=7.40 Viscosity=510,000 cps (Brookfield Model RVT, TE @ 5 rpm).

Alterations can be made in the above without departing from the scope of this invention.

1. A personal care composition for treatment of hair or skin comprising:
(i) a cosmetically acceptable medium comprising at least one cosmetically acceptable additive selected from the group consisting of conditioning agents, protecting agents, fixing agents, oxidizing agents, reducing agents and dyes, and
(ii) a cationic polymer derived from:
(a) at least one vinyllactam or alkylvinyllactam monomer;
(b) at least one monomer having the structure 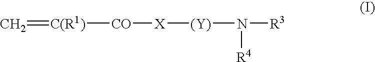
and
(c) at least one quaternized monomer having the structure 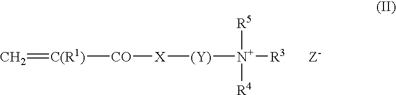
wherein X is an oxygen atom or an NR⁶ radical;
R¹ and R⁶ are each independently H or C₁-C₅ alkyl;
R³, R⁴ and R⁵ are each independently H or C₁-C₃₀ alkyl;
Y is an alkylene radical having from 2 to 16 carbon atoms and
Z is an anion of an organic or mineral acid.

2. The composition of claim 1 wherein said vinyllactam or alkylvinyllactam monomer is a compound of structure (IV): 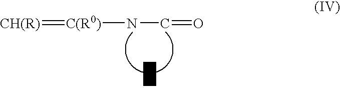
in which:
P is C₃ to C₆ alkylene optionally substituted with lower alkyl,
R and R^{o} are independently H or a C₁-C₅ alkyl, provided that at least one of R and R⁰ is H.

3. The composition of claim 2 wherein said vinyllactam monomer is vinylpyrrolidone.

4. The composition of claim 1 wherein R³, R⁴, and R⁵ are independently H or C₁-C₃₀ linear or branched alkyl.

5. The composition of claim 1 wherein said anion Z⁻ is selected from the group consisting of halide ions, phosphate ions, methosulfate ion and tosylate ion.

6. The composition of claim 1 wherein said cationic polymer further comprises one or more cationic or nonionic monomers.

7. The composition of claim 1 wherein said cationic polymer contains, by weight, 40 to 95% monomer (a), 0.25 to 50% monomer (b), and 0.1 to 55% monomer (c).

8. The composition of claim 1 wherein said cationic polymer is a terpolymer selected from the group consisting of vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, vinylpyrrolidone/dimethylaminopropylmethacrylamide/lauryldimethyl-methacrylamidopropylammonium tosylate or chloride terpolymers.

9. The composition of claim 1 wherein said cationic polymer has a weight average molecular weight between 200,000 and 2,000,000.

10. The composition of claim 1 wherein said cationic polymer is present in an amount from about 0.01 to 10 wt % of the total weight of the composition.

11. The composition of claim 10 wherein said cationic terpolymer is present in an amount from 0.1 to 5 wt % of the total weight of the composition.

12. The composition of claim 1 wherein said additive is a conditioning agent.

13. The composition of claim 12 wherein said conditioning agent is selected from the group consisting of synthesis oils, mineral oils, vegetable oils, fluoridated or perfluoridated oils, natural or synthetic waxes, silicones, cationic polymers other than the cationic polymer defined in claim 1, proteins and hydrolyzed proteins, ceramide type compounds, cationic surfactants, fatty amines, fatty acids and derivatives thereof, and mixtures of said conditioning agents.

14. The composition of claim 13 wherein said conditioning agent is a cationic polymer is selected from the group consisting of polymers containing recurring units consisting of primary, secondary, tertiary and/or quaternary amines, quaternary derivatives of cellulose ether, cationic cyclopolymers, cationic polysaccharides, quaternary polymers of vinyl pyrrolidone and vinyl imidazole, and mixtures thereof.

15. The composition of claim 13 wherein said conditioning agent is a silicone selected from the group consisting of polyalkyl siloxanes, polyaryl siloxanes, polyalkyl aryl siloxanes, silicone gums, resins, and organo-modified silicones.

16. The composition of claim 1 wherein said additive is a protecting agent.

17. The composition of claim 16 wherein said protecting agent is selected from the group consisting of hydrosoluble, liposoluble and water-insoluble UV filters, antiradical agents, antioxidants, vitamins and provitamins.

18. The composition of claim 1 wherein said additive is a fixing agent.

19. The composition of claim 18 wherein said fixing agent is an anionic polymer chosen from polymers containing carboxylic units derived from unsaturated carboxylic mono- or diacids of the formula: 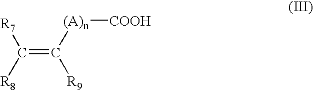
in which n is a whole number from 0 to 10, A₁ denotes a methylene group, optionally bonded to the carbon atom of the unsaturated group or to a neighboring methylene group when n is greater than 1 by means of a heteroatom like oxygen or sulfur, R₇ denotes a hydrogen atom, a phenyl or benzyl group, R₈ denotes a hydrogen atom, a lower alkyl or carboxyl group, R⁹ denotes a hydrogen atom, a lower alkyl group, a —CH₂—COOH, phenyl or benzyl group and polymers containing units derived from sulfonic acid like vinylsulfonic, styrenesulfonic, acrylamidoalkylsulfonic units.

20. The composition of claim 18 wherein said fixing agent is an amphoteric polymer containing units derived from:
a) at least one monomer selected from acrylamides or methacrylamides substituted on the nitrogen with an alkyl radical,
b) at least one acid monomer containing one or more reactive carboxyl groups, and
c) at least one basic monomer, selected from esters with primary, secondary, tertiary, and quaternary amino substituents of acrylic and methacrylic acids and the product of quaternization of dimethylaminoethyl methacrylate with dimethyl or diethyl sulfate.

21. The composition of claim 18 wherein said fixing agent is a nonionic polymer selected from polyalkyloxazolines; vinyl acetate homopolymers; vinyl acetate and acrylic ester copolymers; vinyl acetate and ethylene copolymers; vinyl acetate and maleic ester copolymers; polyethylene and maleic anhydride copolymers; homopolymers of alkyl acrylates; homopolymers of alkyl methacrylates; copolymers of acrylic esters; copolymers of alkyl acrylates and alkyl methacrylates; copolymers of acrylonitrile and a nonionic monomer chosen from among butadiene and alkyl(meth)acrylates; copolymers of alkyl acrylate and urethane; and polyamides.

22. The composition of claim 18 wherein said fixing agent is a functionalized or unfunctionalized silicone or non-silicone polyurethane.

23. The composition of claim 18 wherein said fixing agent is a polymer of the grafted silicone type containing a polysiloxane portion and a portion consisting of a nonsilicone organic chain, with one of the two portions forming the main chain of the polymer, and with the other being grafted onto said main chain.

24. The composition of claim 18 wherein said fixing agent is present in the composition in a relative weight concentration between 0.1 and 10%.

25. The composition of claim 1 wherein said additive is an oxidizing agent.

26. The composition of claim 25 wherein said oxidizing agent is selected from the group consisting of hydrogen peroxide, urea peroxide, alkali metal bromates or ferricyanides, persalts, and redox enzymes, optionally with their respective donor or cofactor.

27. The composition of claim 26 wherein said oxidizing agent is hydrogen peroxide.

28. The composition of claim 27 characterized in that said composition is a solution of oxygenated water whose titer varies from 1 to 40 volumes.

29. The composition of claim 28 characterized in that said composition has a pH greater than 7.

30. The composition of claim 1 wherein said additive is a reducing agent.

31. The composition of claim 30 wherein said reducing agent is selected from the group consisting of thiols, thioglycolic acid, thiolactic acid and cysteamine.

32. The composition of claim 1 wherein said additive is a dye.

33. The composition of claim 32 wherein said dye is selected from the group consisting of neutral acid or cationic nitrobenzene dyes; neutral acid or cationic azo dyes; quinone dyes; neutral, acid or cationic anthraquinone dyes; azine dyes; triarylmethane dyes; indoamine dyes and natural dyes.

34. The composition of claim 32 wherein said dye is present in an amount from 0.001 to 20 wt % of the total weight of the composition.

35. The composition of claim 1 further comprising at least one amphoteric polymer or cationic polymer different from the cationic poly(vinyllactam) defined in claim 1.

36. The composition of claim 35 wherein said cationic polymer is a poly(quaternary ammonium) consisting of recurrent units corresponding to the following formula (W):

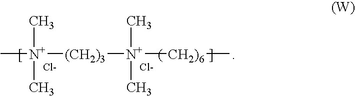

37. The composition of claim 35 wherein said cationic polymer is a poly(quaternary ammonium) consisting of recurrent units corresponding to the following formula (U):

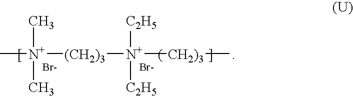

38. The composition of claim 35 wherein said amphoteric polymer is a copolymer containing at least one acrylic acid and a dimethyldiallyammonium salt as a monomer.

39. The composition of claim 35 wherein said cationic or amphoteric polymer or polymers are present in an amount of 0.01 to 10 wt % of the total weight of the composition.

40. The composition of claim 1 further comprising at least one surfactant chosen from anionic, cationic, nonionic or amphoteric surfactants.

41. The composition of claim 40 wherein said surfactant is present in an amount of 0.01 to 40 wt % of the total weight of the composition.

42. The composition of claim 1 characterized in that said composition has a pH from 2 to 12.

43. The composition of claim 1 wherein said cosmetically acceptable medium is formed from water or one or more cosmetically acceptable solvents.

44. The composition of claim 1 further comprising at least one additive selected from the group consisting of cleansing agents, anionic, cationic, nonionic, or amphoteric surfactants, perfumes, stabilizers, pH adjusters, preservatives, colorants, bleaching agents, highlighting agents sequestrants and any other additive conventionally used in cosmetic compositions.

45. The composition of claim 1 further comprising at least one alkalizing or acidifying agent in an amount from 0.01 to 30 wt % of the total weight of the composition.

46. The composition of claim 45 wherein said alkalizing agent is selected from the group consisting of ammonia, alkali carbonates, alkanolamines, mono-, di- and triethanolamines and derivatives thereof, hydroxyalkylamines, ethoxylated and/or propoxylated ethylenediamines, sodium or potassium hydroxides and compounds of the following formula (XIX): 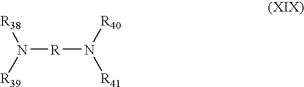
in which R is a propylene residue optionally substituted with an hydroxyl group or a C₁-C₄ alkyl radical; R₃₈, R₃₉, R₄₀ and R₄₁, identical or different, represent a hydrogen atom, a C₁-C₄ alkyl radical or a C₁-C₄ hydroxyalkyl radical.

47. The composition of claim 45 wherein said acidifying agent is selected from mineral or organic acids, carboxylic and sulfonic acids.

48. The composition of claim 1 wherein said composition is anhydrous and intended for bleaching or permanent wave waving of human keratin fibers.

49. The composition of claim 1 which is a skin altering composition and which further contains a cosmetic removal agent, a skin stimulating agent, a depilatory agent, an astringent, a skin abrading agent, a wrinkle mollifying agent, a moisturizing agent, a sun blocking agent, a skin soothing or healing balm and mixtures thereof.
